(19) United States
(12) Patent Application Publication
(10) Pub. No.: US 2021/0210185 A1
(43) Pub. Date: Jul. 8, 2021

(54) SYSTEMS AND METHODS FOR AUTOMATED PROCESSING OF MEDICAL PRESCRIPTIONS

(71) Applicant: RxThat, Inc., Castle Rock, CO (US)

(72) Inventors: James Allred, Alvaton, KY (US); Brian N. Cunningham, Castle Rock, CO (US); Jason Cole, Louisville, CO (US); Vishal Patel, Austin, TX (US); Adam Mamelak, Austin, TX (US)

(21) Appl. No.: 17/142,791

(22) Filed: Jan. 6, 2021

Related U.S. Application Data
(60) Provisional application No. 62/957,661, filed on Jan. 6, 2020.
(60) Provisional application No. 63/093,422, filed on Oct. 19, 2020.

(51) Int. Cl. G16H 20/10; G16H 40/67; G06Q 20/08

(52) U.S. Cl. CPC G16H 20/10; G06Q 20/0855; G16H 40/67

(57) ABSTRACT

A system for processing medical prescriptions is disclosed. The system comprises computer hardware in communication with a customer, a prescriber, and a plurality of pharmacies. The computer hardware includes a processor and a storage medium including instructions for execution by the processor to receive insurance information from the customer and receive a medical prescription and prescription-related rules from the prescriber. The processors obtain a customer cost associated with fulfilling the medical prescription at each pharmacy based on the insurance information and communicate the customer costs to the customer. The customer provides pharmacy selection input (e.g., one or more preferred pharmacies) and the processors transmit the medical prescription to a selected pharmacy for fulfillment based on the prescription-related rules (e.g., a list of approved pharmacies).

### CROSS-REFERENCE TO RELATED APPLICATIONS

This application claims the benefit of priority to U.S. Provisional Application No. 62/957,661 entitled “Systems and Methods for Automated Processing of Medical Prescriptions,” filed Jan. 6, 2020, and U.S. Provisional Application No. 63/093,422 entitled “Systems and Methods for Automated Processing of Medical Prescriptions,” filed Oct. 19, 2020, each of which is incorporated herein by reference in its entirety.

### TECHNICAL FIELD

The present disclosure relates generally to methods and systems related to processing and fulfilling medical prescriptions. More particularly, the present disclosure relates to methods and systems for improved interfacing and automated communications between prescribers, patients, pharmacies, and pharmaceutical companies.

### BACKGROUND

Prescription medication therapy is well-known as a standard component of medical treatment for many patients. Along with the advancement of modern medicine, the increasing availability and efficacy of medications for the treatment of acute and chronic conditions and as preventative measures has resulted in prescription medication therapy playing an increasing role in patient care.

Despite these advances, many patients encounter various obstacles to receiving prescription medication in a simple, efficient, and cost-effective manner. Typically, in order to obtain prescribed drugs, a patient is diagnosed by a physician as having a condition requiring medication, and the physician prepares a prescription for an appropriate medication. The prescription is communicated to a selected pharmacy at which the patient may fulfill the medication. In many cases, the patient may encounter various obstacles upon corresponding with or even arriving at the selected pharmacy. For example, the cost of the prescription medication may vary drastically among pharmacies. As such, the cost at the selected pharmacy may be unexpectedly and/or excessively high. Further, in many cases equivalent medications such as generic equivalents or clinically similar medications may be available at lower prices as compared to a prescribed medication. Further complicating the issue, coverage afforded by health insurance and prescription benefits may vary greatly between a prescribed medication and equivalent medications. Additionally, the acceptance of different forms of insurance may vary from pharmacy to pharmacy, thus affecting the pricing available to the consumer at each location and, ultimately, the effective price to fulfill the prescription. Furthermore, patients are not able to comparison shop between applying insurance and paying full cash price. In many cases, the cost to the patient may be lower if a patient does not apply his or her insurance. Because patients would not typically have access to real-time pricing across pharmacies or knowledge of equivalent medications, patients may be unaware of more cost-effective options.

Even when and where patients are able to identify more convenient or economical fulfillment options, several barriers may prevent modification of a prescription and/or transfer of the prescription to a different pharmacy. For example, once a medical prescription is received at a pharmacy, regulations in some states require a person-to-person phone call between pharmacies in order to complete a transfer of the medical prescription. As such, automation of prescription transfers through electronic systems is difficult and time-consuming. Patients may alternatively contact the prescriber to seek a re-issue of the medical prescription to a different pharmacy. In either case, however, direct human interaction is currently required, thus lengthening or even hindering the process altogether. Similarly, modifications to the medical prescription such as equivalent drug substitution (EDS) requires authorization from the prescriber. In many cases, prescriptions may be abandoned and never filled due to these barriers.

Due to the various obstacles present in the prescription fulfillment process, patients often delay or forego medical prescription therapy, which can result in suboptimal treatment. Further, prescribers cannot readily determine whether prescribed medications are fulfilled and adhered to, making it more difficult to evaluate prescribed treatments.

As such, it would be advantageous to have a system that provides real-time pricing of medications across pharmacies while accounting for the patient's prescription benefits in order to optimize pricing for the patient. It would be further advantageous to automate various modifications to fulfilling medical prescriptions in order to simplify the fulfillment process and provide feedback to prescribers regarding adherence to prescribed treatments.

### SUMMARY

This summary is provided to comply with 37 C.F.R. § 1.73. It is submitted with the understanding that it will not be used to interpret or limit the scope or meaning of the present disclosure.

A system for processing medical prescriptions is provided. The system comprises one or more processors in communication with a customer, a prescriber, and a plurality of pharmacies; and a non-transitory, computer-readable medium storing instructions that, when executed, cause the one or more processors to: receive insurance information from the customer; receive one or more prescription-related rules from the prescriber; receive a medical prescription for a medication from the prescriber; obtain, for each pharmacy of the plurality of pharmacies, a customer cost associated with fulfilling the medical prescription based on the insurance information; communicate the customer cost for each pharmacy to the customer; receive a pharmacy selection input from the customer; and transmit the medical prescription to a selected pharmacy of the plurality of pharmacies based on the pharmacy selection input and the one or more prescription-related rules.

According to some embodiments, the one or more processors are further in communication with a cost evaluation module, and obtaining a patient cost associated with fulfilling the medical prescription comprises transmitting the insurance information to the cost evaluation module, and receiving the customer cost from the cost evaluation module.

According to some embodiments, the customer cost received from the cost evaluation module is based further on discount information. According to additional embodiments, the instructions, when executed, further cause the one or more processors to: receive discount information associated with the customer; and adjust the customer cost for the pharmacy based on the discount information. According to additional embodiments, the discount information is related to one or more of a pharmacy benefit manager, a pharmacy membership, a discount program, an employment benefit program, a co-pay card, a discount card, and a coupon. According to additional embodiments, the discount information is received from one or more of the customer and the pharmacy.

According to some embodiments, the one or more processors communicate with the customer through a customer application on a remote customer computing device.

According to some embodiments, the one or more processors communicate with the prescriber through a prescriber application on a remote prescriber computing device.

According to some embodiments, the one or more processors communicate with each pharmacy through a pharmacy application on a remote pharmacy computing device.

According to some embodiments, receiving a medical prescription for a medication from the prescriber comprises: receiving possession of the medical prescription at a routing entity associated with the system; and receiving prescription information associated with the medical prescription at the one or more processors.

According to some embodiments, receiving a medical prescription for a medication from the prescriber comprises: receiving possession of the medical prescription at a holding pharmacy associated with the system; and receiving prescription information associated with the medical prescription at the one or more processors.

A method of processing a medical prescription is also provided. The method comprises: receiving, from a prescriber, a medical prescription for a customer; receiving, from the customer, insurance information related to the customer; determining, for each pharmacy of a plurality of pharmacies, a customer cost associated with fulfilling the medical prescription based on the insurance information; receiving a pharmacy selection input from the customer; and transmitting, based on the pharmacy selection input, the medical prescription to a selected pharmacy of the plurality of pharmacies.

According to some embodiments, receiving one or more prescription-related rules from the prescriber, and transmitting the medical prescription to a selected pharmacy is further based on the one or more prescription-related rules.

According to some embodiments, receiving discount information associated with the customer; and adjusting the customer cost for the pharmacy based on the discount information. According to additional embodiments, the discount information is related to one or more of a pharmacy benefit manager, a pharmacy membership, a discount program, an employment benefit program, a co-pay card, a discount card, and a coupon. According to additional embodiments, the discount information is received from one or more of the customer and the pharmacy.

According to some embodiments, receiving a medical prescription for a customer comprises: receiving possession of the medical prescription at a routing entity; and receiving prescription information associated with the medical prescription at one or more processors. According to additional embodiments, transmitting the medical prescription to a selected pharmacy comprises directly transferring the possession of the medical prescription from the routing entity to the selected pharmacy.

According to some embodiments, receiving a medical prescription for a customer comprises: receiving possession of the medical prescription at a holding pharmacy; and receiving prescription information associated with the medical prescription at one or more processors. According to additional embodiments, transmitting the medical prescription to a selected pharmacy comprises: transferring the possession of the medical prescription from the holding pharmacy to the prescriber; and transferring the possession of the medical prescription from the prescriber to the selected pharmacy.

### BRIEF DESCRIPTION OF THE DRAWINGS

The accompanying drawings, which are incorporated in and form a part of the specification, illustrate the embodiments of the invention and together with the written description serve to explain the principles, characteristics, and features of the invention. In the drawings:

FIG. 1 depicts a block diagram of an illustrative network for processing a medical prescription in accordance with an embodiment.

FIG. 2 depicts an illustrative information flow diagram for a prescription processing system in accordance with an embodiment.

FIG. 3 depicts an illustrative flow diagram of a method of processing a medical prescription in accordance with an embodiment.

FIG. 4 depicts an illustrative flow diagram of a method of transferring a medical prescription in accordance with an embodiment.

FIG. 5 depicts an illustrative flow diagram of a method of substituting a medication of a medical prescription in accordance with an embodiment.

FIG. 6 illustrates a block diagram of an illustrative data processing system in which aspects of the illustrative embodiments are implemented.

FIG. 7 depicts a block diagram of an illustrative network for processing a medical prescription in accordance with an alternate embodiment.

FIGS. 8A-8C depict illustrative views of an interface of a patient module 115 displayed on a display of a mobile device in accordance with an embodiment.

### DETAILED DESCRIPTION

This disclosure is not limited to the particular systems, devices and methods described, as these may vary. The terminology used in the description is for the purpose of describing the particular versions or embodiments only, and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

For the purposes of this disclosure, the terms “subject,” “patient,” and “user” are used interchangeably and as used herein include a person receiving or registered to receive prescription medication therapy.

For the purposes of this disclosure, it should also be understood that the term “customer” may be interchangeable with “subject,” “patient,” and/or “user.” For example, a medical patient may utilize the systems and methods described herein to fulfill a medical prescription for herself. However, it should also be understood that in some instances, the customer may not be the patient. For example, a customer may be an individual that utilizes the systems and methods described herein to fulfill a medical prescription for the patient, e.g., a family member, friend, employer, employee, and the like. The customer may use the patient's information to fulfill the prescription in this context. It should be understood that the systems and methods described herein contemplate situations where the customer and the patient may be distinct individuals and are equally applicable therein.

As discussed herein, in the course of medical treatment, a prescriber (e.g., a physician) may issue a medical prescription to a patient and transmit the medical prescription to a pharmacy for fulfillment. In many cases, in order to improve efficiency and accuracy, the prescriber may utilize computer-based systems for generating and transmitting the medical prescription to the pharmacy (i.e., e-prescribing). Further, various additional interactions may be required on the part of the prescriber in the process of fulfillment. For example, approval may be required in order to transfer the medical prescription to a different pharmacy or substitute the prescribed medication or formulation.

The systems and methods described herein are intended to simplify and optimize fulfillment of medications. The described systems and methods may provide real-time pricing access to patients and/or prescribers, thereby decreasing realized costs to patients and increasing trust and transparency. Prescribers and pharmacies may also benefit from greater efficiency (e.g., automated prescription transfers and/or modifications) and increased patient satisfaction. Pharmacies may further benefit from greater visibility and decreased customer acquisition costs. Additionally, the described systems and methods provide greater communication and information between prescribers, patients, and pharmacies. The described systems and methods may further increase the likelihood of initiating prescribed medication therapies, increase adherence to prescribed medication therapies, decrease the rate of abandonment of prescribed medication therapies, and decrease delay in receiving medications.

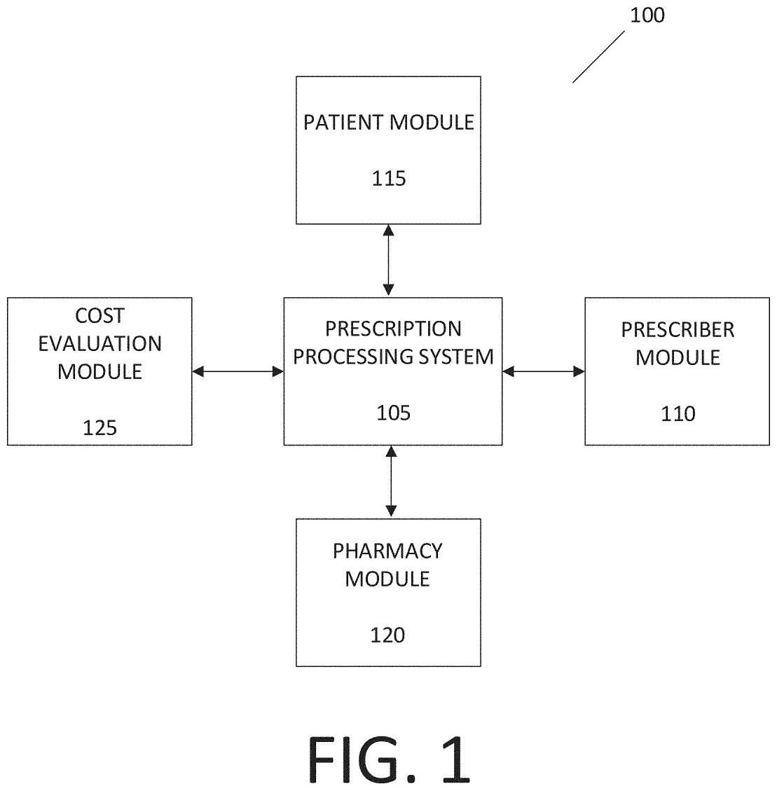

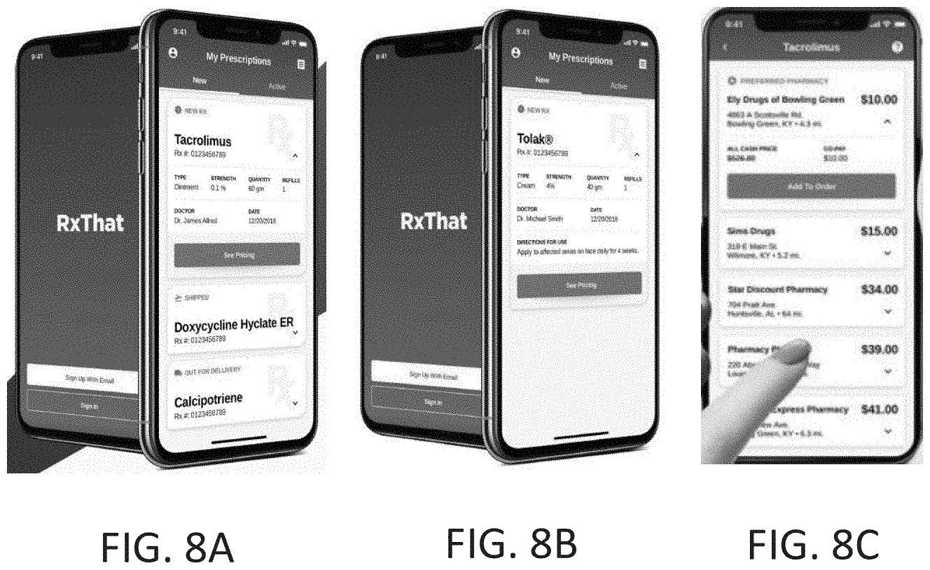

Referring now to FIG. 1, an illustrative block diagram of a network 100 for processing a medical prescription is depicted in accordance with an embodiment. As shown, the network 100 comprises a prescription processing system 105 including one or more processors that serves as a central hub for communicating data between one or more external modules. For example, as shown in FIG. 1, the prescription processing system 105 may be in communication with a prescriber module 110, which may be referred to as a virtual medical assistant (VMA). The prescription processing system 105 may further be in communication with a patient module 115 (also referred to as a customer module), such as a mobile device application (see, e.g., FIGS. 8A-8C). The prescription processing system 105 may be further in communication with a pharmacy module 120, such as a pharmacist portal. The prescription processing system 105 may be further in communication with a cost evaluation module 125, such as a claim switch. Each of the external modules may comprise an application (i.e., a software application) on a computing device remote from the prescription processing system. For example, each external module can be hosted on a mobile device, a tablet, a personal computer, or other computing device. Through the network 100, the prescription processing system 105 and the external modules may transmit data to one another and/or receive data from one another.

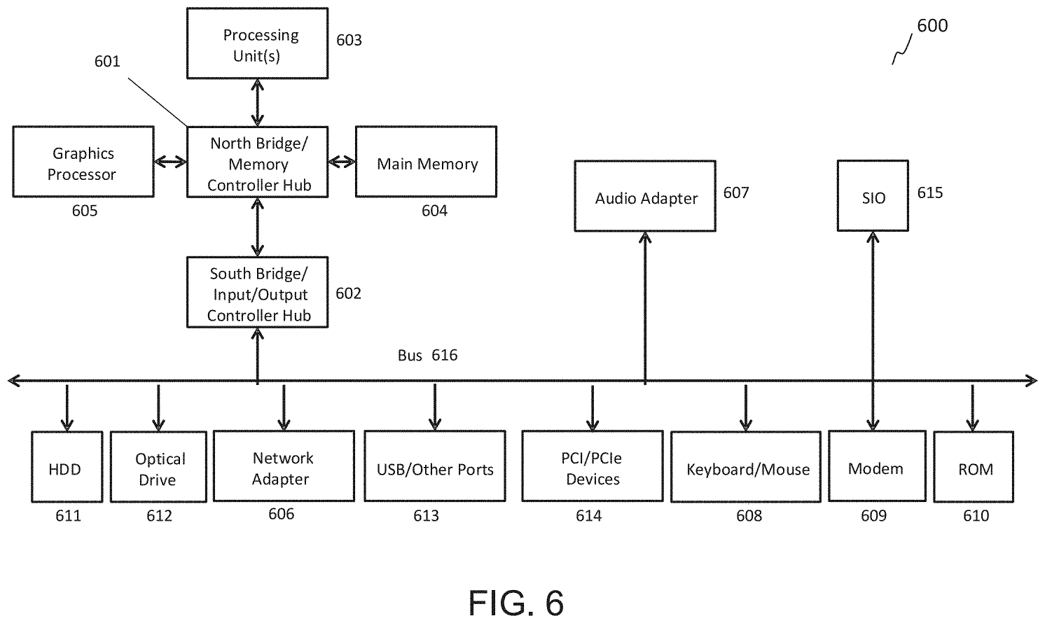

The prescription processing system 105 may take any number of forms. In some embodiments, the system 105 may comprise a server such as a network server, a remote server, a plurality of servers at the same or remote locations, a virtual server, or a physical server. In further examples, the system 105 may comprise a mainframe, a host computer, a workstation, or any other computing device operable to perform the functions described herein. The structure and components of an exemplary system are described herein with respect to FIG. 6.

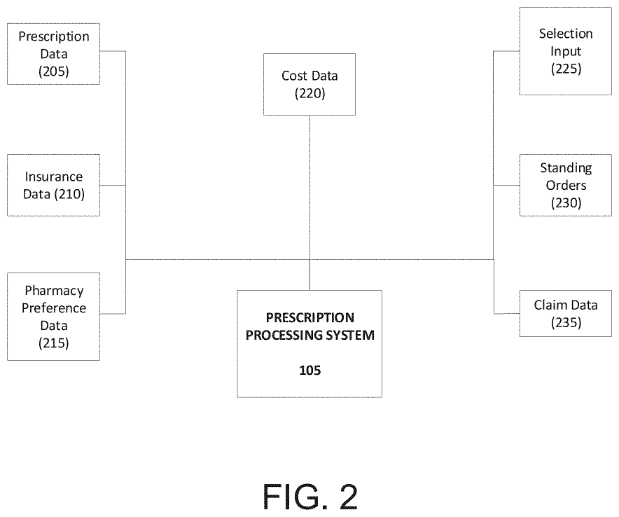

FIG. 2 depicts an information flow diagram for the network 100 according to an embodiment. As shown in FIG. 2, the system 105 may receive and/or store various types of data 205-230 as described herein from the external modules. Further, the system 105 may transmit the various types of data 205-230 to any of the external modules.

In some embodiments, the system 105 is configured to receive and transmit prescription data 205 related to a medical prescription for a patient, which may comprise a variety of information. For example, the medical prescription may specify a medication being prescribed. The medical prescription may further indicate a dosage form of the medication, including but not limited to a particular strength of the medication, a preparation type (e.g., tablet, capsule, syrup, solution, cream, ointment, drops, suppository, and the like), and an indication of acceptability of generic substitutes. In some embodiments, the medical prescription may include a total amount of the medication, a dose, a number of administrations, a frequency of administration, a route of administration (e.g., oral, buccal, intravenous, intramuscular, intranasal, subcutaneous, topical, inhalation, rectal, and the like), and a number of refills. In some embodiments, the medical prescription may further include additional instructions regarding administration. For example, the prescriber may specify how to take the medication, such as taking with food or taking upon rising. In a further example, the prescriber may specify criteria for taking the medication, such as in response to one or more symptoms. In additional embodiments, the medical prescription may include patient-identifying information such as a name and/or a date of birth. In still additional embodiments, the medical prescription may include prescriber-identifying information such as a name, an address, a practice name, and/or an ID number (e.g., an NPI number or a DEA number). Additional information not explicitly described herein may be included in the prescription data 205 as is known to one having an ordinary level of skill in the art. In some embodiments, prescription data 205 is received from a prescriber (e.g., a physician) via the prescriber module 110. However, it is contemplated that the prescription data 205 may be acquired, either together or separately, through a variety of sources. For example, in some cases, a pharmacy and/or a patient may receive prescription data 205 directly from a physician and transmit to the system 105 through a pharmacy module 120 and/or a patient module 115.

In some embodiments, the system 105 is configured to receive and transmit insurance data 210 for a patient, which may comprise a variety of information. For example, the insurance data 210 may include gender, a zip code, an RxBIN, an RxPCN, a Group ID, a Member ID, a Person Code, a Patient Relationship Code, a member ID, a policy number, and/or a group number. In some embodiments, the insurance data 210 includes prescription benefits, such as a formulary (i.e., a list of prescription drugs covered by the insurance company), a pharmacy network, and/or prescription costs or tiers. In further embodiments, the insurance data 210 includes information related to a benefits contract. The benefits contract may include details regarding various coverages, including but not limited to coverage amounts (e.g., percentages or dollar amounts of coverage associated with different tiers/groups of medications) and/or associated co-pays. In still additional embodiments, insurance data 210 may include identifying information for the primary insured individual and/or dependents, such as a name, a date of birth, and/or a relationship to the primary insured individual. Additional information not explicitly described herein may be included in the insurance data 210 as is known to one having an ordinary level of skill in the art. In some embodiments, the insurance data 210 is received from the patient via a patient module 115. However, it is contemplated that the insurance data 210 may be acquired, either together or separately, through a variety of sources. For example, in some cases, the insurance data 210 may be received by the system 105 via a pharmacy module 120 and/or a prescriber module 110.

In some embodiments, the system 105 is configured to receive and transmit pharmacy preference data 215 for a patient indicative of one or more potential locations for fulfilling the medical prescription. A pharmacy preference may depend on a variety of factors, including but not limited to distance, travel time, convenience, accessibility, a patient's comfort, a patient's familiarity, and/or a patient's trust associated with a particular location. Thus, in some embodiments, the pharmacy preference data 215 may include input from a patient indicating one or more pharmacies (i.e., one or more specific locations) as preferred pharmacies. In some embodiments, the pharmacy preference data 215 may alternatively or additionally include one or more geographical locations and/or a proximity to one or more geographical locations. For example, the pharmacy preference data 215 may include one or more locations such as a recent (e.g., current) geographical location of the patient, a home address, a work address, previously visited pharmacy locations, a currently selected pharmacy, and/or additional locations of interest for the patient. The pharmacy preference data 215 may also include a proximity threshold, such as a maximum distance and/or travel time in conjunction with the one or more locations. As such, even without explicit indication by the patient of one or more preferred pharmacies, the system 105 may use the available data to deduce likely preferential pharmacy locations. The system 105 may utilize any of the pharmacy preference data 215 described herein to determine a set of preferred pharmacies for the patient. It is also contemplated that additional preferences and/or additional factors not explicitly described herein may be considered and included in the pharmacy preference data 215 as is known to one having an ordinary level of skill in the art. Pharmacy preference data 215 may include indications with respect to individual pharmacies and/or with respect to groups of pharmacies. In some cases, the patient may select one or more pharmacies individually. Alternatively or additionally, the patient may select a group, such as a brand or chain of pharmacies, as preferred locations. In some embodiments, the pharmacy preference data 215 is received from a patient via a patient module 115. However, it is contemplated that the pharmacy preference data 215 may be acquired, either together or separately, through a variety of sources. In some cases, the pharmacy preference data 215 may be received by the system 105 via a prescriber module 110. For example, a physician may indicate a pharmacy or pharmacies for fulfillment based on consultation with the patient. The pharmacy preference data 215 may include the specifically indicated pharmacies, and may further include the geographical locations of the specifically indicated pharmacies such that additional nearby pharmacies may be identified.

In some embodiments, the system 105 is configured to receive cost data 220 associated with a medical prescription. For example, the system 105 may be configured to receive a cost to the patient associated with fulfilling a medical prescription at a particular pharmacy. In some embodiments, the system 105 may be configured to transmit data to the cost evaluation module 125. The system 105 may transmit any data received or stored therein, including but not limited to the prescription data 205, insurance data 210, and pharmacy preference data 215. For example, the cost evaluation module may be a claim switch and the transmitted data may include required fields for submitting a claim. In response, the cost evaluation module 125 may transmit to the system 105, for each of one or more pharmacies, cost data 220 associated with fulfilling the medical prescription (i.e., a cost to the patient). The system may use various programs or application programming interfaces to receive the cost data. Further, in some embodiments, the cost data 220 may include a plurality of patient costs for each of the one or more pharmacies. For example, for each of the one or more pharmacies, the cost evaluation module 125 may transmit a patient cost associated with fulfilling the medical prescription through an insurance claim as well as one or more patient costs associated with fulfilling the medical prescription without an insurance claim (e.g., purchasing through a pharmacy membership program, a discount program, purchasing at full cash price, and/or purchasing with one or more coupons). Further, the system 105 may be configured to adjust the patient costs based on information from additional sources, e.g. coupon information associated with a pharmacy or a pharmacy benefit manager (PBM). While some or all of the cost data 220 may be received from the cost evaluation module 125, it is contemplated that the cost data 220 may be acquired, either together or separately, through a variety of sources. In some cases, a portion of the cost data 220 may be received from a pharmacy module 120. For example, a full cash price associated with fulfilling the medical prescription without utilizing insurance may be received from a pharmacy module 120 based on available pricing at a particular pharmacy. In some embodiments, cost data 220 may additionally or alternatively be generated by the prescription processing system 105 to provide additional available pricing for fulfilling a medical prescription. For example, the prescription processing system may utilize a formula or algorithm to derive a price (e.g., based on a formulary as described herein) available to customers through the prescription processing system which is different from the price offered through insurance coverage and/or by one or more pharmacies.

In some embodiments, the system 105 is further configured to receive and transmit a selection input 225 from a patient. For example, the selection input 225 may comprise a pharmacy selection input, e.g., a selected pharmacy for fulfilling the medical prescription which is selected from the one or more potential locations. Selection may be based on preference as described herein, as well as costs associated with fulfilling the medical prescription at each of the one or more potential locations. In another example, the selection input 225 may comprise a mode of purchase, e.g., a selected mode of purchase from a plurality of available modes of purchase such as a full cash purchase, a purchase through insurance, a purchase through a pharmacy membership program, a purchase through a discount program, and/or a purchase with one or more coupons. In some embodiments, the selection input 225 is received from the patient via a patient module 115. However, it is contemplated that the selection input 225 may be acquired through a variety of sources.

In some embodiments, the system 105 is configured to receive one or more standing orders or rules 230 (also referred to as prescription-based rules). While prescribers generate medical prescriptions with specific instructions, in some cases it may be necessary or desirable to modify the medical prescription in various manners. As such, the standing orders 230 may indicate acceptable modifications or unacceptable modifications to the medical prescription. In some embodiments, a standing order 230 may indicate a list of acceptable (i.e., trusted) pharmacies for fulfillment of the medical prescription. For example, the list of acceptable pharmacies may be a national directory of licensed pharmacies in good standing. In other embodiments, the list of acceptable pharmacies may be further limited for any of a variety of reasons. In further embodiments, a standing order 230 may indicate whether a substitution of the medication is acceptable. In another example, the standing order 230 may address substitution of one strength of the medication for another strength of the medication. In one example, the standing order 230 may address substitution of a brand name medication for a generic equivalent medication. In another example, the standing order 230 may address substitution of one preparation or dosage form for another preparation or dosage form of the medication (e.g., substituting an ointment for a cream). In another example, the standing order 230 may address substitution of the medication for another clinically similar medication (e.g., substituting a topical steroid such as clobetasol for another topical steroid such as halobetasol). In some embodiments, one or more standing orders 230 may be general rules associated with a prescriber. In other embodiments, one or more standing orders 230 may be specific to a medical prescription for a particular patient. While exemplary standing orders are described, additional types of standing orders are contemplated herein and may be included in the standing orders 230 as is known to one having an ordinary level of skill in the art. In some embodiments, the standing orders 230 are received from the prescriber via a prescriber module 110. However, it is contemplated that the standing orders 230 may be acquired, either together or separately, through a variety of sources. In some cases, one or more standing orders 230 may also be derived by the system 105 based upon the available information. For example, where the prescription information 205 includes an indication of the acceptability of generic equivalents, the system 105 may derive a standing order specific to the medical prescription which addresses generic equivalent substitution.

In some embodiments, the system 105 is configured to receive claim data 235. For example, a cost evaluation module may provide, in addition to cost data 220, additional information related to a potential claim related to the medical prescription. In some embodiments, the claim data 235 comprises requirements for coverage associated with the medical prescription (e.g., requirement to fill at a mail order pharmacy). In some embodiments, the claim data 235 comprises a reason for denial or ineligibility. For example, the claim data 235 may include information regarding particular classes or types of medication which are not covered, information related to why a particular patient is not covered, information related to eligibility periods, information related to required authorizations, information related to requirements for a specialty pharmacy, information related to quantity limits, information related to refill coverage and/or refill time period, information related to step therapy programs, information related to insurance networks, and the like. In some embodiments, the claim data 235 comprises a reason for not evaluating a claim. For example, the claim data 235 may include indications of incorrect or invalid data, non-responsive insurance systems, lack of coverage, and the like. In some embodiments, the claim data 235 comprises one or more instructions or requests for additional information in order to better evaluate insurance coverage and/or provide cost data. For example, the claim data 235 may include a request for re-submission with a brand product (e.g., a product preferred by a payor), a request for re-submission with a different product (e.g., a product preferred by a payor with a lower co-pay), a request for submission to another payor, a request for Drug Utilization Review (DUR) outcome code, a request for an ICD-10 (International Classification of Diseases) diagnosis code, and the like. In some embodiments, claim data 235 is received from the cost evaluation module 125. However, it is contemplated that the claim data 235 may be acquired, either together or separately, through a variety of sources.

In addition to the types of data described with respect to FIG. 2 and depicted therein, it is contemplated that various additional types of data may be received and/or transmitted by the system 105. In some embodiments, the system 105 may receive and/or transmit patient records, physician's notes, and any other documentation related to the patient in order to facilitate communication between pharmacies, clinic staff, and patients. In some embodiments, the system 105 may receive and/or transmit prescription monitoring information such as historical records of written (i.e., issued) and/or filled medical prescriptions in order to better inform prescribers of patient adherence to recommended treatment. In some embodiments, the system 105 may receive and/or transmit status information for medication orders, including but not limited to delivery status, estimated delivery time, and estimated “ready for pick-up” time. In some embodiments, the system 105 may receive and/or transmit inventory or purchase history (e.g., including wholesale costs of medications) from one or more pharmacies or databases.

It should be understood that various types of information as described herein may be shared from the system 105 to various external modules in additional manners. For example, the system 105 may be configured to share prescription data 205, insurance data 210, cost data 220, and/or claim data 235 with a patient via the patient module 115. In some embodiments, various types of data may be displayed on the patient module 115 and may be used to elicit information from the patient. For example, FIGS. 8A-8C depict illustrative views of an interface of a patient module 115 displayed on a display of a mobile device in accordance with an embodiment. In some embodiments, selection input 225 may be received from the patient via the patient module 115 in response to insurance data 210 and the cost data 220 that is communicated to the patient via the patient module 115, e.g., by displaying information on a display of a computing device such as a mobile device. In a particular example, one or more patient costs associated with fulfilling the medical prescription may be displayed to the patient via the patient module 115 and a selection input 225 may be received from the patient based on the one or more patient costs. In some embodiments, the patient module 115 may display one or more patient costs associated with each of a plurality of pharmacies (e.g., as shown in FIG. 8C). For example, the patient may view and compare costs for fulfilling the prescription at several pharmacies on the patient module (e.g., a side-by-side comparison). In some embodiments, the patient module 115 may display one or more patient costs associated with a plurality of modes of purchase. For example, the patient may view and compare costs for fulfilling the prescription through several available modes of purchase on the patient module (e.g., a side-by-side comparison) including but not limited to full cash purchase, purchase through insurance, purchase through pharmacy membership program, purchase through discount program, and/or purchasing with one or more coupons. Accordingly, selection input 225 and/or other types of input from the patient may be based on data provided to the patient via the patient module.

In another example, the system 105 may provide a schedule or timeline associated with a prescribed treatment plan to a patient through the patient module 115. Accordingly, the patient may view the schedule along with reminders and notifications for administration of the prescription through the patient module. Further, information associated with the schedule or timeline may be provided to the system 105 by the patient via the patient module 115. For example, a user may provide input to indicate administration of the medication at specific times, thereby providing a record of adherence to a treatment plan including completed doses and/or missed doses.

Additional types of data as described herein may be provided to the patient via the patient module 115. In some embodiments, the system 105 may communicate advertisements to the patient via the patient module 115. In some embodiments, the system 105 may communicate educational information associated with a medication to the patient via the patient module 115, e.g., instructions for administering the medication, warnings associated with the medication and/or additional information commonly provided with the medication. In some embodiments, the educational information may comprise text, graphics, photos, and/or videos. In some embodiments, the system 105 may communicate reminders or notifications associated with adherence via the patient module 115. For example, the system 105 may communicate reminders or notifications within the software application, reminders or notifications on the operating system of the computing device (i.e., push notifications), and/or reminders through additional modes of communication (e.g., text message reminders or notifications). Reminders and/or notifications may increase adherence to a treatment plan by the patient, thus providing improved outcomes and/or valuable therapy response information that may be communicated to a prescriber. In some embodiments, the system 105 may communicate information related to a therapy response (e.g., information associated with monitoring of one or more symptoms).

While the communication of various types of information is described above with respect to the system 105 and the patient module 115, it should be understood that information may be shared from the system 105 to the prescriber module 110, the pharmacy module 120, and/or the cost evaluation module 125 in a similar manner as would be apparent to a person having an ordinary level of skill in the art. Furthermore, the various types of information and/or input related thereto may be received by the system 105 from any of the modules 110-125 in a similar manner.

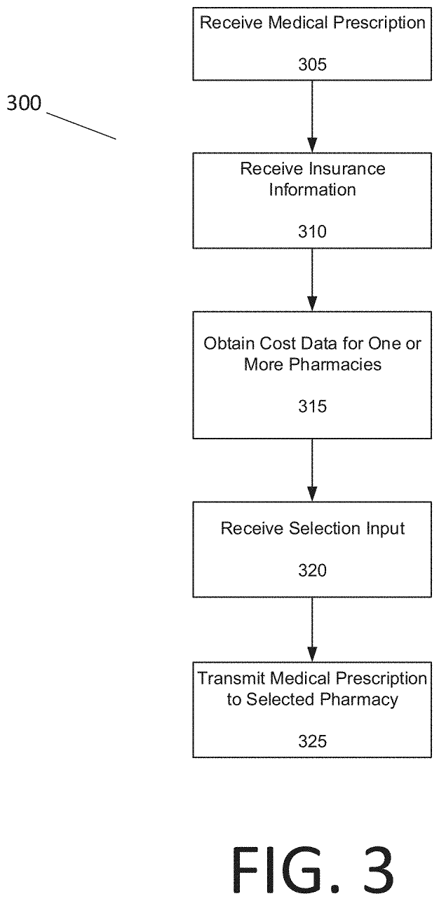

FIG. 3 depicts a flow diagram 300 of an illustrative method of processing a medical prescription by a processing system (e.g., prescription processing system 105 of FIG. 1) through a network (e.g., network 100 of FIG. 1) in accordance with an embodiment. As shown in FIG. 3, a medical prescription for a patient is received 305 from a prescriber through a prescriber module (e.g., prescriber module 110 as described herein) and insurance information is received 310 from the patient through a patient module (e.g., patient module 115 as described herein). Based on the prescription and the insurance information, cost data associated with fulfilling the prescription is obtained 315 through a cost evaluation module (e.g., cost evaluation module 125 as described herein) for each of one or more pharmacies. In some embodiments, the prescription and insurance information or portions thereof are transmitted to the cost evaluation module, and the patient costs are received in response thereto. Selection input related to the one or more pharmacies is received 320 from the patient module, and the medical prescription is transmitted 325 to the selected pharmacy for fulfillment via a pharmacy module (e.g., pharmacy module 120 as described herein).

As discussed herein, receiving the medical prescription 305 may comprise receiving possession of the medical prescription at an entity associated with the processing system. For example, the processing system may be or may include a holding pharmacy or other entity that takes possession of the medical prescription. Furthermore, receiving the medical prescription 305 may further comprise receiving prescription information associated with the medical prescription for processing.

In some embodiments, the prescription processing system may utilize available information to identify the one or more pharmacies for which to obtain cost data and to present to the patient for selection. For example, the prescription processing system may receive pharmacy preference information and the one or more pharmacies for which cost data is obtained may be based on the pharmacy preference information. The prescription processing system may also further evaluate the pharmacy selection information and/or the selection input against one or more standing orders or prescription-based rules from the prescriber. For example, the selection input may be evaluated against a list of acceptable pharmacies for fulfillment in order to ensure that the selected pharmacy is approved by the prescriber. Alternatively, the system may utilize the list in addition to the pharmacy selection information in order to identify the one or more pharmacies for which to obtain cost data, thus ensuring that the selected pharmacy will be an approved pharmacy.

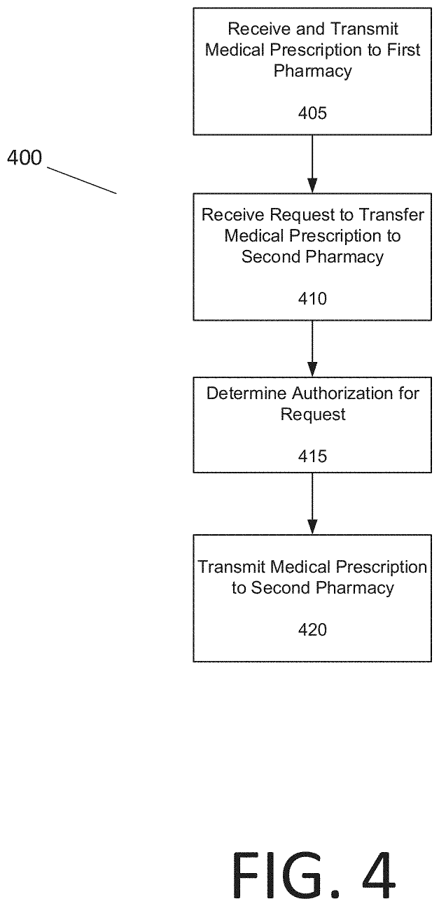

FIG. 4 depicts a flow diagram 400 of an illustrative method of transferring a medical prescription in accordance with an embodiment. As shown in FIG. 4, a medical prescription may be received 405 (e.g., at a holding pharmacy or other entity) from a prescriber module. In some embodiments, the medical prescription may be received at a first pharmacy. In some embodiments, a prescription processing system 105 includes the first pharmacy because an entity associated with the prescription processing system 105 may be registered as a pharmacy such that prescribers may transmit medical prescriptions thereto. Accordingly, as discussed herein, receiving the medical prescription 405 may comprise receiving possession of the medical prescription at the first pharmacy and/or receiving prescription information associated with the medical prescription. In some embodiments, the prescription processing system 105 may be or may include a registered pharmacy that may take possession of medical prescriptions but solely re-routes the medical prescriptions to other pharmacies, i.e., a “holding pharmacy” that does not fulfill the medical prescriptions itself. In other embodiments, the prescription processing system 105 may be a non-pharmacy entity configured to route prescriptions to a pharmacy (i.e., a clearinghouse). A request related to transferring the medical prescription to a second pharmacy may be received 410 from a patient module. In some embodiments, the request may be based on selection input. For example, the patient may select, via the patient module, a second pharmacy for fulfillment. A determination of whether the request has authorization may be made 415 based on one or more standing orders or rules from a prescriber module (i.e., from the prescriber issuing the medical prescription). In some embodiments, the one or more standing orders or rules may include a list of acceptable pharmacies for fulfillment as described herein. Where authorization for the second pharmacy exists, the medical prescription may be transmitted 420 to the second pharmacy via a pharmacy module. In the absence of proper authorization, the process may be halted, and the prescription may remain with the first pharmacy.

In some embodiments, multiple requests related to transferring the medical prescription may be received. For example, in some cases the medical prescription may be transmitted to a second pharmacy for fulfillment based on selection by the patient, and the patient may subsequently select, via the patient module, a third pharmacy for fulfillment that is different from the second pharmacy. In another example, the second pharmacy may provide selection input via the pharmacy module based on consultation with the patient and/or prescriber (e.g., if the prescription cannot be filled at the first pharmacy).

Further, in the absence of proper authorization, a denial notification may be sent to the patient module. In some embodiments, the patient may be prompted to select a third pharmacy for which a request may be transmitted, and the process may repeat until an authorized pharmacy is selected. In other embodiments, the system may utilize the one or more standing orders or rules (e.g., the list of acceptable pharmacies) to identify and present one or more authorized pharmacies to the patient for selection of the second pharmacy, thus ensuring that the second pharmacy will be an approved pharmacy.

In some embodiments, the medical prescription may be transmitted 420 to the second and/or third pharmacy direct from the prescription processing system via a pharmacy module. In other embodiments, the medical prescription may be routed through the prescriber module for transmission to the second and/or third pharmacy.

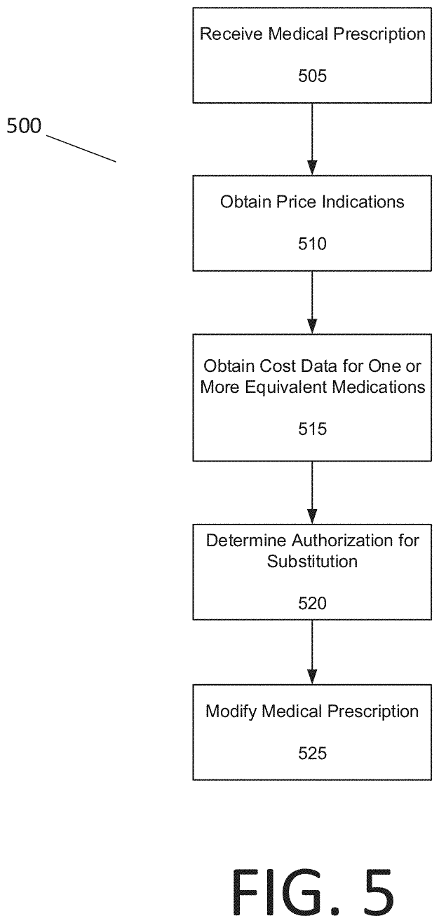

FIG. 5 depicts a flow diagram 500 of an illustrative method of substituting a medication of a medical prescription in accordance with an embodiment. As shown in FIG. 5, a medical prescription for a prescribed formulation is received 505 (e.g., at a holding pharmacy or other entity) from a prescriber via a prescriber module and one or more price indications are obtained 510. In some embodiments, the price indications may include wholesale costs for the prescribed formulation and/or equivalent formulations obtained from pharmacy modules or other databases. In some embodiments, the price indications may include price data related to historical medical prescriptions for the prescribed formulation and/or equivalent formulations. In some embodiments, the price indications may include copay costs for the prescribed formulation and/or equivalent formulations obtained from the claim switch, pharmacy modules or other databases. In some embodiments, the price indications may include pharmaceutical company copay assistance cards, coupons, discount cards or other related tools to reduce the patient's out-of-pocket costs for the medication and/or equivalent medications obtained from the claim switch, pharmacy modules or other databases, In some embodiments, the price indications may include a denial letter (or other such notification) received from a claim switch for the medical prescription. For example, a denial letter (or other such notification) may indicate one or more formulations that are not covered by a patient's prescription benefits and/or one or more equivalent formulations that are covered by the patient's prescription benefits. In some embodiments, the price indications may include information from a benefits contract related to various medication coverages. Based on the price indications, cost data for one or more equivalent medications is obtained 515. In some embodiments, the cost data for each of the one or more equivalent medications is obtained from a cost evaluation module and/or through a claim switch. In some embodiments, the cost data is determined based on price data related to historical medical prescriptions. Further, a determination of whether substitution of the prescribed formulation with the one or more equivalent formulations has authorization is made 520 based on one or more standing orders or rules from a prescriber module (i.e., from the prescriber issuing the medical prescription) or within the prescription processing system. In some embodiments, the one or more standing orders or rules may include a list of acceptable equivalent formulations. Where authorization for the substitution exists, the medical prescription is modified 525 to substitute the prescribed formulation with the equivalent formulation. In the absence of proper authorization, the substitution may be declined, and the medical prescription may remain in its initial form.

In some embodiments, the prescription processing system may store a formulary including a plurality of medications. The formulary may also include information related to one or more available strengths of a medication, one or more preparation types or dosage forms of a medication, and other prescription information as discussed herein. The prescription processing system may also store one or more substitution rules describing substitution of a first medication of the formulary for a second medication of the formulary. In some embodiments, a substitution rule may indicate an acceptable substitution. In some embodiments, a substitution rule may indicate an unacceptable substitution. As such, the method 500 may be performed by additionally or alternatively consulting the formulary and substitution rules in order to evaluate authorization for a substitution. In some embodiments, the method 500 may be performed without consulting a prescriber module for standing orders or rules (i.e., relying on the formulary and substitution rules in order to evaluate the authorization for a substitution). In some embodiments, the formulary and substitution rules may be utilized in place of or in addition to the one or more price indications to identify one or more equivalent medications for which to obtain cost data.

In some embodiments, in the absence of proper authorization, one or more additional equivalent medications may be identified and the patient cost may be obtained 515 therefor. Thereafter, authorization for the substitution may be determined 520. The process may repeat until an authorized equivalent medication is identified. In other embodiments, the system may utilize the one or more standing orders or rules (e.g. the list of acceptable pharmacies) to identify and present one or more equivalent medications prior to determining patient costs, thereby ensuring that the equivalent medications are approved by the prescriber.

The devices, systems, and methods as described herein are not intended to be limited in terms of the particular embodiments described, which are intended only as illustrations of various features. Many modifications and variations to the devices, systems, and methods can be made without departing from their spirit and scope, as will be apparent to those of ordinary skill in the art.

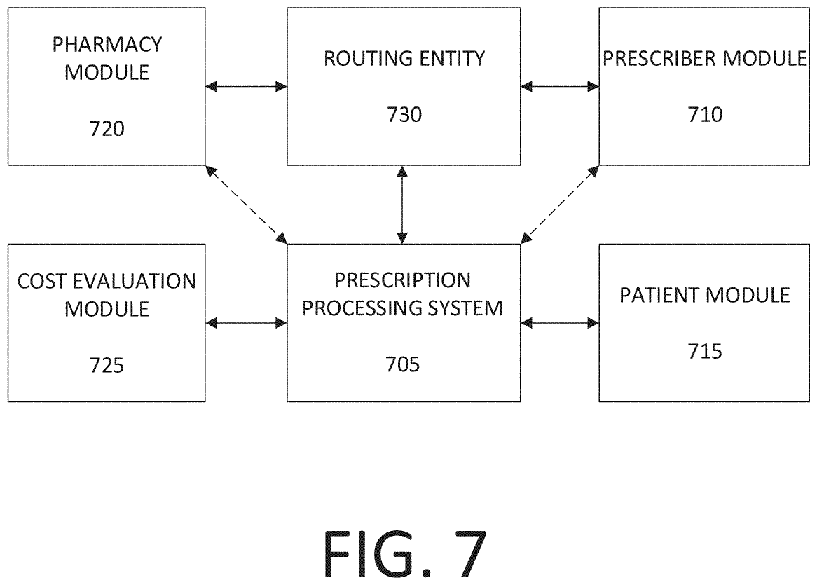

Referring now to FIG. 7, an illustrative block diagram of a network 700 for processing a medical prescription is depicted in accordance with an alternate embodiment. Similar or corresponding features between network 100 of FIG. 1 and network 700 of FIG. 7 are identified with common reference numbers. Similar to the network 100, the network 700 comprises a prescription processing system 705 including one or more processors that serve as a central hub for communicating data between one or more external modules. For example, as shown in FIG. 7, the prescription processing system 705 may be in communication with a patient module 715 and/or a cost evaluation module 725. Furthermore, the prescription processing system 705 may be in communication with a routing entity 730 configured to directly receive a prescription from a prescriber module 710 and directly transmit the prescription to a pharmacy module 720. Through the prescription processing system 705 and the routing entity 730, the external modules may transmit data to one another and/or receive data from one another.

As discussed herein, the prescription processing system 705 may be prohibited from directly transferring a prescription electronically to a pharmacy. For example, as discussed herein, the prescription processing system 705 may be registered as a pharmacy (e.g., a “holding pharmacy”) and may thus be subject to jurisdiction-specific regulations for transfers of prescriptions between pharmacies. Accordingly, the routing entity 730 may interface with the prescriber module 710 to receive possession of the medical prescription therefrom and retain the prescription until a pharmacy is identified for transmission. The routing entity 730, as a non-pharmacy entity, may then transfer the prescription into the possession of the selected pharmacy (i.e., the corresponding pharmacy module 720).

While the routing entity 730 retains possession of the prescription, the prescription processing system 705 may nonetheless receive various types of data (e.g., data 205-235 as described herein) and identify a pharmacy for transfer of the prescription. The routing entity 730 may share prescription information with the prescription processing system 705. The prescription processing system 705 may transmit instructions to the routing entity 730 to transfer the prescription to the selected pharmacy in order to complete the transaction.

In some embodiments, the routing entity 730 may transmit information to the prescription processing system 705 in order to facilitate the methods 300, 400, 500 and additional functions of the prescription processing system 705 as described herein. For example, the routing entity 730 may receive prescription data 205, standing orders or rules 230, and/or additional types of data as described herein from the prescriber module 710 and may transmit the data, in whole or in part, to the prescription processing system 705. Furthermore, the routing entity 730 may receive cost data 220 and/or additional types of data as described herein from the pharmacy module 720 and may transmit the data, in whole or in part, to the prescription processing system 705. The prescription processing system 705 may use the data to perform any of the various functions described herein, including but not limited to selection of a pharmacy for transfer of the prescription.

In some embodiments, the prescription processing system 705 may not directly interface with the prescriber module 710. Accordingly, the prescription processing system 705 may only exchange information with the prescriber module 710 through the routing entity 730. However, in some embodiments, the prescription processing system 705 may interface with the prescriber module 710 (depicted in FIG. 7 as a broken line). Thus, except as it pertains to transfer of the prescription, the prescription processing system 705 may receive information directly from the prescriber module 710 and/or transmit information thereto in the manner described herein with respect to the prescription processing system 105 of FIG. 1.

In some embodiments, the prescription processing system 705 may not directly interface with the pharmacy module 720. Accordingly, the prescription processing system 705 may only exchange information with the pharmacy module 720 through the routing entity 730. However, in some embodiments, the prescription processing system 705 may interface with the pharmacy module 720 (depicted in FIG. 7 as a broken line). Thus, except as it pertains to a transfer of the prescription, the prescription processing system 705 may receive information directly from the pharmacy module 720 and/or transmit information thereto in the manner described herein with respect to the prescription processing system 105 of FIG. 1.

In some embodiments, the network 700 may handle prescriptions in various manners based on the jurisdictional restrictions applicable to each transaction. In some transactions, the routing entity 730 may receive the prescription from the prescriber module 710 and transfer the prescription to the pharmacy module 720 under instruction from the prescription processing system 705 (e.g., in jurisdictions where pharmacy-to-pharmacy electronic transfers of prescriptions are prohibited). In other transactions, the prescription processing system 705 may receive the prescription directly from the prescriber module 710 and directly transfer the prescription to the pharmacy module 720 (e.g., in jurisdictions where pharmacy-to-pharmacy electronic transfers of prescriptions are permitted), thereby completing the transfer without involvement of the routing entity 730. In additional transactions, the prescription processing system 705 may receive the prescription directly from the prescriber module 710 and transfer the prescription to the routing entity 730. The routing entity 730 may then transfer the prescription to the pharmacy module 720 under instruction from the prescription processing system 705.

It should be understood that the various methods described herein (e.g., methods 300, 400, and 500) may be performed by the network 700 in substantially the same manner as the network 100 as described herein and/or with minor modifications as would be understood to a person having an ordinary level of skill in the art. For example, the methods 300, 400, and/or 500 may be modified in instances where the prescription transfer is facilitated through the routing entity 730.

With respect to the method 300 of FIG. 3, in some embodiments, receiving 305 the medical prescription comprises receiving the medical prescription at the routing entity 730. In some embodiments, obtaining 315 cost data for one or more pharmacies comprises obtaining cost data at the prescription processing system 705 via the routing entity 730. In some embodiments, transmitting 325 the medical prescription to the selected pharmacy comprises transmitting the medical prescription from the routing entity 730.

With respect to the method 400 of FIG. 4, in some embodiments, receiving 405 the medical prescription at a first pharmacy comprises receiving the medical prescription from the routing entity 730. In some embodiments, determining 415 authorization for a request comprises determining authorization based on one or more standing orders or rules received from a prescriber module via the routing entity 730. In some embodiments, transmitting 420 the medical prescription to a second pharmacy comprises transmitting the medical prescription from the routing entity 730.

With respect to the method 500 of FIG. 5, in some embodiments, receiving 505 the medical prescription comprises receiving the medical prescription at the routing entity 730. In some embodiments, obtaining 510 price indications comprises obtaining one or more price indications from a pharmacy module via the routing entity 730. In some embodiments, obtaining 515 cost data for one or more equivalent medications comprises obtaining costa data from a pharmacy module via the routing entity 730. In some embodiments, determining 520 authorization for a substitution comprises determining authorization based on one or more standing orders or rules received from a prescriber module via the routing entity 730. In some embodiments, modifying 525 the medical prescription comprises modifying the medical prescription by the routing entity 730.

In some embodiments, the prescription processing systems 105 and/or prescription processing system 705 may be in communication with one or more prescriber modules, one or more patient modules, one or more pharmacy modules, and/or one or more cost evaluation modules. For example, it is contemplated that an external module may be dedicated for each prescriber, each patient, and/or each pharmacy. The external modules generally comprise one or more processors; however, they may take a variety of forms. Each of the external modules may comprise a computer, a server, a portable or mobile device, and/or an application for these or other computing devices known to one having an ordinary level of skill in the art. For example, the prescriber module and pharmacy module may comprise an application on a desktop or laptop computer, while the patient module may comprise an application on a mobile device. FIGS. 8A-8C depict illustrative views of the application displayed on a mobile device in accordance with an embodiment. In some embodiments, additional external modules may communicate with the prescription processing system in order to transmit information thereto and receive information therefrom. In some embodiments, the prescription processing system may be in communication with one or more external databases which provide information such as wholesale costs of medications, cost of medications based on historical prescription data, coverage-related information based on historical prescription data, and the like. For example, if coverage for a particular medication is regularly denied by a particular healthcare insurance provider, the system may utilize this information to more efficiently identify cost-effective substitutions. In some embodiments, one or more of the described external modules may be eliminated from the ecosystem or combined with the prescription processing system. For example, the prescription processing system may effectively determine patients costs based on historical data or information related to a patient's benefits contract, thus rendering a separate cost evaluation module unnecessary. In some embodiments, one or more functions attributed to an external module may be re-assigned to another external module and/or performed by the prescription processing system. For example, the prescription processing system may receive patient costs with or without utilizing prescription benefits from an external cost evaluation module. Then, the prescription processing system may utilize additional data to further modify the patient costs, such as applying available co-pay coupons, discounts, or other cost-saving means.

While various interactions are described as transmitting or receiving particular information between various components (i.e., the prescription processing system, external modules, and/or routing entity), the nature of the interaction in any and all steps described herein may take different forms. In some cases, a component may transmit a request for particular information from another component and receive a transmission of information in response thereto. In some cases, information may be transmitted unprompted (e.g., upon entry or receipt of updated information at one of the external modules). In some embodiments, the prescription processing system may receive data relevant to a required determination or evaluation such that the prescription processing system may make a required determination or evaluation. However, in other embodiments, the prescription processing system may transmit additional relevant data such that the external module and/or routing entity can make the required determination or evaluation. For example, in determining whether a medication substitution is authorized by the prescriber, the prescription processing system may receive one or more standing orders and evaluate the medication substitution against the one or more standing orders. Alternatively, the prescription processing system may transmit the proposed medication substitution to the prescriber module and the prescriber module may transmit authorization or denial in response thereto. In some embodiments, the prescription processing system may not include a storage medium and may request and receive relevant data in real-time as required for processing a medical prescription. In other embodiments, the prescription processing system includes a storage medium such that it may receive and store one or more types of data and access the stored data when relevant to a processing a medical prescription. The stored data may be checked periodically and confirmed or updated as necessary. For example, the prescription processing system may receive and store a prescriber's standing orders and check for updates thereto at set intervals.

While the steps of the various methods herein are described in a particular sequence, it is contemplated that any of the various steps may be performed in a different sequence. For example, the system may evaluate authorization of various parameters (e.g., one or more pharmacies for selection by the patient or one or more equivalent medications for substitution) prior to presenting options for selection to ensure that the selected option has authorization.

While the methods 300, 400, 500 are described herein as individual processes, it is contemplated that any of the methods could be performed simultaneously in any combination. As the methods are integrated into a singular process, one or more steps as described herein may be omitted. Further, in some embodiments, processing of a single medical prescription may comprise performing one or more of the methods repeatedly. For example, upon generation of a medical prescription by a prescriber, the system may perform one or more steps of the method 300 to determine patient costs at one or more pharmacies. In this process, the system may receive price indications according to the method 500 and trigger evaluation of one or more equivalent medications, resulting in modification of the medical prescription. Further, at any point it may be determined that the selected pharmacy for fulfillment (e.g., selected by the patient or the prescriber) is no longer ideal, and the network 100 or the network 700 may transfer the medical prescription to a second pharmacy according to the method 400. Any of these methods may be omitted and/or repeated throughout the processing of the medical prescription. For example, in some embodiments, a single medical prescription may comprise a plurality of medications and as a result, optimal patient cost may be achieved by fulfilling each medication at a separate pharmacy. As such, various steps of the methods may be repeated for each medication of the medical prescription. In other embodiments, all medications of the medical prescription may be fulfilled at a single pharmacy location. In some embodiments, the prescriber may not specify a pharmacy for fulfillment of the medical prescription and input may be obtained from the patient in order to select a pharmacy prior to an initial transmission. In other embodiments, a prescriber may transmit the medical prescription to a first pharmacy (with or without consultation with the patient), and the patient may request transfer of the prescription through the prescription processing system if a different pharmacy is preferred.

In some embodiments, the prescription processing system and/or other components of the network may be an artificial intelligence (AI) system which develops foundational knowledge based on historical data sets. Historical data sets (e.g., historical price data from previously processed medical prescriptions) may be used as inputs to a machine learning model such as, for example, a recurrent neural network (RNN) or other form of artificial neural network. As is generally understood in the art, an artificial neural network functions similar to a biologic neural network and is comprised of a series of nodes and connections. The machine learning model is trained to predict one or more values based on the input data. In some embodiments, the AI system may be trained to predict pricing for a prescribed medication, equivalent medications, and variances therebetween. In some embodiments, the AI system may be trained to interpret benefits contracts and/or denial letters in order to identify patient costs for medications, covered and uncovered equivalent medications, and the like. In further embodiments, any functions of the system and/or steps of the methods as described herein may be further automated or optimized by the AI system.

The systems and methods described herein may further incorporate various additional functions. In some embodiments, a patient may be able to purchase, submit payment, and schedule delivery for a prescription medication through the patient module in communication with the prescription processing system. In some embodiments, the patient may be able to bundle the purchase of prescription medications with non-prescription items. For example, where a pharmacy is included in a larger retailer or department store, a patient may combine the purchase and delivery of prescription medications with non-prescription medication, grocery or other retail items. In some embodiments, a patient may be able to utilize co-pay cards and/or rewards programs through the patient module.

In some embodiments, the network 100 and/or network 700 may provide additional functions for prescribers. In some embodiments, prescribers may be able to track prescription fulfillment by patients in order to evaluate compliance. In some embodiments, prescribers may also be able to view patients' prescription histories in order to inform evaluation. In some embodiments, prescribers may be provided with prescribing metrics in order to evaluate the value of particular prescriptions. For example, a prescriber may be able to visualize the rate at which prescriptions for a particular medication are covered by insurance or fulfilled by the patient. A prescriber may be able to compare metrics for a particular medication to known equivalent medications in order to improve the cost-effectiveness and/or fulfillment rate of prescriptions.

The systems and methods described herein may be utilized with respect to various types of medical practices. In some embodiments, the prescriber may be a physician (e.g., a dermatologist). However, the prescriber may be of any specialty and may be a medical doctor, an osteopathic doctor, a nurse practitioner, a physician's assistant, a naturopathic doctor, a dentist, an optometrist, a pharmacist, a podiatrist, a veterinarian, or any other professional authorized to prescribe medication.

FIG. 6 illustrates a block diagram of an illustrative data processing system 600 in which aspects of the illustrative embodiments are implemented. The data processing system 600 is an example of a computer, such as a server or client, in which computer usable code or instructions implementing the process for illustrative embodiments of the present invention are located. In some embodiments, the data processing system 600 may be a server computing device. For example, data processing system 600 can be implemented in a server or another similar computing device operably connected to ecosystem of external modules as described above. The data processing system 600 can be configured to, for example, transmit and receive information related to a patient, a related medical prescription, and/or a related medication.

In the depicted example, data processing system 600 can employ a hub architecture including a north bridge and memory controller hub (NB/MCH) 601 and south bridge and input/output (I/O) controller hub (SB/ICH) 602. Processing unit 603, main memory 604, and graphics processor 605 can be connected to the NB/MCH 601. Graphics processor 605 can be connected to the NB/MCH 601 through, for example, an accelerated graphics port (AGP).

In the depicted example, a network adapter 606 connects to the SB/ICH 602. An audio adapter 607, keyboard and mouse adapter 608, modem 609, read only memory (ROM) 610, hard disk drive (HDD) 611, optical drive (e.g., CD or DVD) 612, universal serial bus (USB) ports and other communication ports 613, and PCI/PCIe devices 614 may connect to the SB/ICH 602 through bus system 616. PCI/PCIe devices 614 may include Ethernet adapters, add-in cards, and PC cards for notebook computers. ROM 610 may be, for example, a flash basic input/output system (BIOS). The HDD 611 and optical drive 612 can use an integrated drive electronics (IDE) or serial advanced technology attachment (SATA) interface. A super I/O (SIO) device 615 can be connected to the SB/ICH 602.

An operating system can run on the processing unit 603. The operating system can coordinate and provide control of various components within the data processing system 600. As a client, the operating system can be a commercially available operating system. An object-oriented programming system, such as the Java™ programming system, may run in conjunction with the operating system and provide calls to the operating system from the object-oriented programs or applications executing on the data processing system 600. As a server, the data processing system 600 can be an IBM® eServer™ System® running the Advanced Interactive Executive operating system or the Linux operating system. The data processing system 600 can be a symmetric multiprocessor (SMP) system that can include a plurality of processors in the processing unit 603. Alternatively, a single processor system may be employed.

Instructions for the operating system, the object-oriented programming system, and applications or programs are located on storage devices, such as the HDD 611, and are loaded into the main memory 604 for execution by the processing unit 603. The processes for embodiments described herein can be performed by the processing unit 603 using computer usable program code, which can be located in a memory such as, for example, main memory 604, ROM 610, or in one or more peripheral devices.

A bus system 616 can be comprised of one or more busses. The bus system 616 can be implemented using any type of communication fabric or architecture that can provide for a transfer of data between different components or devices attached to the fabric or architecture. A communication unit such as the modem 609 or the network adapter 606 can include one or more devices that can be used to transmit and receive data.

Those of ordinary skill in the art will appreciate that the hardware depicted in FIG. 6 may vary depending on the implementation. Other internal hardware or peripheral devices, such as flash memory, equivalent non-volatile memory, or optical disk drives may be used in addition to or in place of the hardware depicted. Moreover, the data processing system 600 can take the form of any of a number of different data processing systems, including but not limited to, client computing devices, server computing devices, tablet computers, laptop computers, telephone or other communication devices, personal digital assistants, and the like. Essentially, data processing system 600 can be any known or later developed data processing system without architectural limitation.

While various illustrative embodiments incorporating the principles of the present teachings have been disclosed, the present teachings are not limited to the disclosed embodiments. Instead, this application is intended to cover any variations, uses, or adaptations of the present teachings and use its general principles. Further, this application is intended to cover such departures from the present disclosure as come within known or customary practice in the art to which these teachings pertain.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by 1/10 of the stated values, e.g., ±10%. The term “about” also refers to variations that would be recognized by one skilled in the art as being equivalent so long as such variations do not encompass known values practiced by the prior art. Each value or range of values preceded by the term “about” is also intended to encompass the embodiment of the stated absolute value or range of values. Whether or not modified by the term “about,” quantitative values recited in the present disclosure include equivalents to the recited values, e.g., variations in the numerical quantity of such values that can occur, but would be recognized to be equivalents by a person skilled in the art.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

What is claimed is:

1. A system for processing medical prescriptions, the system comprising:
one or more processors in communication with a customer, a prescriber, and a plurality of pharmacies; and
a non-transitory, computer-readable medium storing instructions that, when executed, cause the one or more processors to:
receive insurance information from the customer;
receive one or more prescription-related rules from the prescriber;
receive a medical prescription for a medication from the prescriber;
obtain, for each pharmacy of the plurality of pharmacies, a customer cost associated with fulfilling the medical prescription based on the insurance information;
communicate the customer cost for each pharmacy to the customer;
receive a pharmacy selection input from the customer; and
transmit the medical prescription to a selected pharmacy of the plurality of pharmacies based on the pharmacy selection input and the one or more prescription-related rules.

2. The system of claim 1, wherein the one or more processors are further in communication with a cost evaluation module, and
wherein obtaining a patient cost associated with fulfilling the medical prescription comprises:
transmitting the insurance information to the cost evaluation module, and
receiving the customer cost from the cost evaluation module.

3. The system of claim 1, wherein the customer cost received from the cost evaluation module is based further on discount information.

4. The system of claim 3, wherein the instructions, when executed, further cause the one or more processors to:
receive discount information associated with the customer; and
adjust the customer cost for the pharmacy based on the discount information.

5. The system of claim 3, wherein the discount information is related to one or more of a pharmacy benefit manager, a pharmacy membership, a discount program, an employment benefit program, a co-pay card, a discount card, and a coupon.

6. The system of claim 3, wherein the discount information is received from one or more of the customer and the pharmacy.

7. The system of claim 1, wherein the one or more processors communicate with the customer through a customer application on a remote customer computing device.

8. The system of claim 1, wherein the one or more processors communicate with the prescriber through a prescriber application on a remote prescriber computing device.

9. The system of claim 1, wherein the one or more processors communicate with each pharmacy through a pharmacy application on a remote pharmacy computing device.

10. The system of claim 1, wherein receiving a medical prescription for a medication from the prescriber comprises:
receiving possession of the medical prescription at a routing entity associated with the system; and
receiving prescription information associated with the medical prescription at the one or more processors.

11. The system of claim 1, wherein receiving a medical prescription for a medication from the prescriber comprises:
receiving possession of the medical prescription at a holding pharmacy associated with the system; and
receiving prescription information associated with the medical prescription at the one or more processors.

12. A method of processing a medical prescription comprising:
receiving, from a prescriber, a medical prescription for a customer;
receiving, from the customer, insurance information related to the customer;
determining, for each pharmacy of a plurality of pharmacies, a customer cost associated with fulfilling the medical prescription based on the insurance information;
receiving a pharmacy selection input from the customer; and
transmitting, based on the pharmacy selection input, the medical prescription to a selected pharmacy of the plurality of pharmacies.

13. The method of claim 12, further comprising:
receiving one or more prescription-related rules from the prescriber,
wherein transmitting the medical prescription to a selected pharmacy is further based on the one or more prescription-related rules.

14. The method of claim 12, further comprising:
receiving discount information associated with the customer; and
adjusting the customer cost for the pharmacy based on the discount information.

15. The method of claim 14, wherein the discount information is related to one or more of a pharmacy benefit manager, a pharmacy membership, a discount program, an employment benefit program, a co-pay card, a discount card, and a coupon.

16. The method of claim 14, wherein receiving the discount information comprises receiving the discount information from one or more of the customer and the pharmacy.

17. The method of claim 12, wherein receiving a medical prescription for a customer comprises:
receiving possession of the medical prescription at a routing entity; and
receiving prescription information associated with the medical prescription at one or more processors.

18. The method of claim 17, wherein transmitting the medical prescription to a selected pharmacy comprises directly transferring the possession of the medical prescription from the routing entity to the selected pharmacy.

19. The method of claim 12, wherein receiving a medical prescription for a customer comprises:
receiving possession of the medical prescription at a holding pharmacy; and
receiving prescription information associated with the medical prescription at one or more processors.

20. The method of claim 19, wherein transmitting the medical prescription to a selected pharmacy comprises:
transferring the possession of the medical prescription from the holding pharmacy to the prescriber; and
transferring the possession of the medical prescription from the prescriber to the selected pharmacy.
